(19) United States
(12) Patent Application Publication
(10) Pub. No.: US 2006/0210525 A1
(43) Pub. Date: Sep. 21, 2006

(54) Dispersions

(76) Inventors: Gabriela Calderó Linnhoff, Barcelona (ES); Josep-Lluis Viladot Petit, Barcelona (ES)

Correspondence Address: COGNIS CORPORATION;PATENT DEPARTMENT, 300 BROOKSIDE AVENUE, AMBLER, PA, 19002, US

(21) Appl. No.: 11/178,879

(22) Filed: Jul. 11, 2005

(30) Foreign Application Priority Data
Jul. 10, 2004 (EP) 04016285.1

(51) Int. Cl. A61K 31/74; A01N 27/00

(52) U.S. Cl. 424/78.09

(57) ABSTRACT

The invention relates to new dispersions, especially for underwater paints, containing (a) a microencapsulated anti-fouling component and (b) an organic solvent.

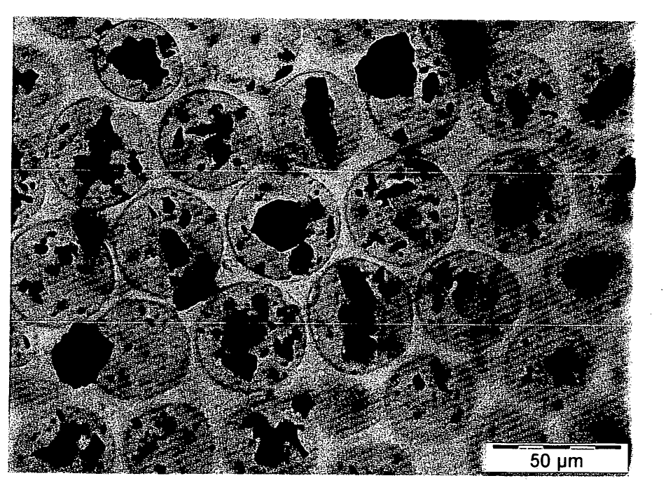

### RELATED APPLICATIONS

This application claims priority from EP 04016285.1 filed Jul. 10, 2004, the entire contents of which is incorporated herein by reference.

### FIELD OF THE INVENTION

This invention relates generally to lacquers and paints and, more particularly, to new dispersions of microencapsulated active components in solvents, to a process for their production and to their use for the production of paints, more particularly for use under water.

### BACKGROUND OF THE INVENTION

Materials are exposed to various factors in the course of their use which can test their resistance. Typical examples are the interplay between wind, rain and temperature differences which, in a comparatively short time, can crack rocks and, over millions of years, can erode entire mountains. Manmade objects are generally not designed to last for such long periods, but even the need to survive for 10, 20 or even 100 years without damage is consistently a challenge to materials technology. A particularly demanding challenge is underwater work in general and the resistance of materials to the effects of seawater in particular. Here, two particularly aggressive factors coincide: on the one hand, the presence of microorganisms, bacteria and marine microorganisms and, on the other hand, the presence of salts. Since the beginnings of seafaring, the first of these two factors in particular has had to be combated time and again by navigators. Whereas, previously, the wooden hull of ships or lake dwellings were mechanically freed from algal growth, special paints known as anti-fouling paints are now used for this purpose [cf. EP 0934368 B1 (Bayer), WO 03/008505 (Foster-Miller)]. If the materials in question are steel or steel-reinforced concrete, the danger of increased corrosion through the gradual penetration of chloride ions is of course another problem that research has attempted to solve by the use of corrosion inhibitors which are added either to the paints or to the concrete.

Despite the many years of experience in the protection of materials by treatment or coating with special protective paints, the results are still unsatisfactory. In particular, the materials are not chemically stable over a sufficiently long period, so that they are unable to perform their protective function for a sufficiently long time. In addition, the stable incorporation of very different materials, such as anti-fouling pigments and corrosion inhibitors for example, in paints and lacquers is problematic.

Accordingly, the problem addressed by the present invention was to provide new preparations with which various materials, such as wood, steel, concrete or even steel-reinforced concrete for example, could be finished or treated in such a way that they would show extended resistance in relation to the prior art both to the effect of microorganisms and to corrosion factors as typically encountered in underwater conditions, especially in seawater.

### BRIEF DESCRIPTION OF THE INVENTION

The present invention relates to dispersions containing

(a) microencapsulated anti-fouling components and

(b) organic solvents.

It has surprisingly been found that, by microencapsulating the anti-fouling components and introducing them into organic solvents, it is possible to produce dispersions which, when added to conventional paints, provide various materials coated or finished with them with improved resistance to various environmental influences, especially underwater and particularly in seawater. The microencapsulation on the one hand protects the materials against chemical decomposition and, on the other hand, releases them with delay, the two effects simultaneously serving the intended purpose of significantly prolonging the period of protection. Another advantage is that, besides the anti-fouling components, the microcapsules may also contain corrosion inhibitors, so that both types of active component may now be used at one and the same time without any formulation difficulties. In this connection, it has been found that, among the various suitable encapsulation systems, gelatin-based microcapsules show particular resistance to seawater which was unexpected and which leads to a distinct preference for the purposes of the present invention.

### DETAILED DESCRIPTION OF THE INVENTION

Anti-Fouling Components

The choice of the anti-fouling components is governed by the particular problem to be addressed—for example whether correspondingly treated ships operate in cold or tropical waters—and, basically, is not critical. Typically, the anti-fouling components are inorganic salts or pigments and/or organometallic compounds. Typical examples are components which contain lead, iron, tin, copper, manganese, arsenic, antimony, bismuth or mercury in particular as their metal constituent, for example copper thiocyanate, copper sulfate, copper pyrithione, zinc ethylene (bis)dithiocarbamate, zinc dimethyl dithiocarbamate, zinc pyrithione, zinc diethyl dithiocarbamate, manganese diethyl dithiocarbamate, the use of oxides of copper and/or iron being preferred. The particles as such generally have a mean diameter of <100 microns (=1.0 mm) and a solubility in water of <100 ppm.

Corrosion Inhibitors

In a preferred embodiment of the present invention, the microcapsules contain not only anti-fouling components, but also corrosion inhibitors. Typical examples are condensation products of C₆₋₂₂ and preferably C₁₂₋₁₈ fatty acids with alkanolamines such as, for example, ethanolamine, propanolamine, diethanolamine, dipropanolamine, triethanolamine, tripropanolamine and the like. Particularly preferred corrosion inhibitors are free from amines, such as for example the coconut oil fatty acid monoethanolamine which is marketed under the name of Texamin® KE 3160 by Cognis. The corrosion inhibitors may be used in quantities of 1 to 100% by weight, preferably 5 to 90% by weight and more particularly 25 to 50% by weight, based on the anti-fouling components.

Organic Solvents

Basically, the choice of the organic solvents is not critical and is largely governed by the regulations of the processing industry. The organic solvents are generally aliphatic, cycloaliphatic or aromatic hydrocarbons, such as for example toluene, xylene or petroleum distillates. The technical mixtures marketed, for example, by Brenntag under the name of Varsol® or by Exxon under the name of Solvesso® are particularly preferred. The dispersions normally contain the microencapsulated active components and the organic solvents in a ratio by weight of 10:90 to 90:10 and preferably 50:50 to 20:80.

Microcapsules and Dispersions Containing Microcapsules

“Microcapsules” are understood by the expert to be spherical aggregates with a diameter of about 0.0001 to about 5 mm and preferably 0.005 to 0.5 mm which contain at least one solid or liquid core surrounded by at least one continuous membrane. More precisely, they are finely dispersed liquid or solid phases coated with film-forming polymers, in the production of which the polymers are deposited onto the material to be encapsulated after emulsification and coacervation or interfacial polymerization. In another process, molten waxes are absorbed in a matrix (“microsponge”) which, as microparticles, may be additionally coated with film-forming polymers. In a third process, particles are coated alternately with differently charged polyelectrolytes (layer-by-layer process). The microscopically small capsules can be dried in the same way as powders. Besides single-core microcapsules, there are also multiple-core aggregates, also known as microspheres, which contain two or more cores distributed in the continuous membrane material. In addition, single-core or multiple-core microcapsules may be surrounded by an additional second, third etc. membrane. The membrane may consist of natural, semisynthetic or synthetic materials. Natural membrane materials are, for example, gum arabic, agar agar, agarose, maltodextrins, alginic acid and salts thereof, for example sodium or calcium alginate, fats and fatty acids, cetyl alcohol, collagen, chitosan, lecithins, gelatin, albumin, shellac, polysaccharides, such as starch or dextran, polypeptides, protein hydrolyzates, sucrose and waxes. Semisynthetic membrane materials are inter alia chemically modified celluloses, more particularly cellulose esters and ethers, for example cellulose acetate, ethyl cellulose, hydroxypropyl cellulose, hydroxypropyl methyl cellulose and carboxymethyl cellulose, and starch derivatives, more particularly starch ethers and esters. Synthetic membrane materials are, for example, polymers, such as polyacrylates, polyamides, polyvinyl alcohol or polyvinyl pyrrolidone.

Examples of known microcapsules are the following commercial products (the membrane material is shown in brackets) Hallcrest Microcapsules (gelatin, gum arabic), Coletica Thalaspheres (maritime collagen), Lipotec Millicapsein (alginic acid, agar agar), Induchem Unispheres (lactose, microcrystalline cellulose, hydroxypropylmethyl cellulose), Unicerin C30 (lactose, microcrystalline cellulose, hydroxypropylmethyl cellulose), Kobo Glycospheres (modified starch, fatty acid esters, phospholipids), Softspheres (modified agar agar), Kuhs Probiol Nanospheres (phospholipids), Primaspheres and Primasponges (chitosan, alginates) and Primasys (phospholipids). Chitosan microcapsules and processes for their production are the subject of earlier patent applications filed in applicants' name [WO 01/01926, WO 01/01927, WO 01/01928, WO 01/01929].

As explained at the beginning, gelatin is particularly suitable for encapsulation of the anti-fouling components and, optionally, the corrosion inhibitors, because the systems on the one hand are easy to produce and, on the other hand, show particularly high stability in a saline environment. Accordingly, they do not dissolve after a short time or possibly even spontaneously, but release the active component slowly over a relatively long period. The microcapsules preferably have a mean diameter of 10 to 150 micras (=0.1 to 1.5 mm).

The dispersions of the microencapsulated active components are generally produced by
(a) preparing a first preparation of an oil component and an emulsifier at ambient temperature,
(b) preparing a second aqueous preparation of gelatin and the active components at a temperature in the range from 50 to 100° C.,
(c) adding the aqueous second preparation dropwise to the first oil-containing preparation with intensive shearing and then cooling to below the melting point of the gelatin,
(d) adding a crosslinking agent to the microcapsule preparations thus obtained,
(e) removing the oil phase and, finally, redispersing the resulting microcapsules in an organic solvent.
   Oil Components

Suitable oil components are, for example, Guerbet alcohols based on fatty alcohols containing 6 to 18 and preferably 8 to 10 carbon atoms, esters of linear C₆₋₂₂ fatty acids with linear C₆₋₂₂ fatty alcohols or esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₁₈₋₃₈ alkyl hydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-/di-/triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, for example Dicaprylyl Carbonate (Cetiol® CC), Guerbet carbonates based on C₆₋₁₈ and preferably C₈₋₁₀ fatty alcohols, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group, for example Dicaprylyl Ether (Cetiol® OE), ring opening products of epoxidized fatty acid esters with polyols, silicone oils (cyclomethicone, silicon methicone types, etc.) and/or aliphatic or naphthenic hydrocarbons, for example squalane, squalene or dialkyl cyclohexanes.

Emulsifiers

Suitable emulsifiers for the oil phase are, for example, nonionic surfactants from at least one of the following groups:
products of the addition of 2 to 30 mol ethylene oxide and/or 0 to 5 mol propylene oxide onto linear C₈₋₂₂ fatty alcohols, C₁₂₋₂₂ fatty acids and alkyl phenols containing 8 to 15 carbon atoms in the alkyl group and alkylamines containing 8 to 22 carbon atoms in the alkyl group;
alkyl and/or alkenyl oligoglycosides containing 8 to 22 carbon atoms in the alkyl group and ethoxylated analogs thereof;
addition products of 1 to 15 mol ethylene oxide onto castor oil and/or hydrogenated castor oil;
addition products of 15 to 60 mol ethylene oxide onto castor oil and/or hydrogenated castor oil;
partial esters of glycerol and/or sorbitan with unsaturated, linear or saturated, branched fatty acids containing 12 to 22 carbon atoms and/or hydroxycarboxylic acids containing 3 to 18 carbon atoms and addition products thereof onto 1 to 30 mol ethylene oxide;
partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and addition products thereof with 1 to 30 mol ethylene oxide;
mixed esters of pentaerythritol, fatty acids, citric acid and fatty alcohol and/or mixed esters of fatty acids containing 6 to 22 carbon atoms, methyl glucose and polyols, preferably glycerol or polyglycerol;
mono-, di- and trialkyl phosphates and mono-, di- and/or tri-PEG-alkyl phosphates and salts thereof;
wool wax alcohols;
polysiloxane/polyalkyl/polyether copolymers and corresponding derivatives;
block copolymers, for example Polyethyleneglycol-30 Dipolyhydroxystearate;
polymer emulsifiers, for example Pemulen types (TR-1), TR-2) from Goodrich;
polyalkylene glycols and
glycerol carbonate.
   Ethylene Oxide Addition Products

The addition products of ethylene oxide and/or propylene oxide onto fatty alcohols, fatty acids, alkylphenols or onto castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of addition products of ethylene oxide onto glycerol are known as lipid layer enhancers for cosmetic preparations.

Alkyl and/or Alkenyl Oligoglycosides

Alkyl and/or alkenyl oligoglycosides, their production and their use are known from the prior art. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based.

Partial Glycerides

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide onto the partial glycerides mentioned are also suitable.

Sorbitan Esters

Suitable sorbitan esters are sorbitan monoisostearate, sorbitan sesqui-isostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxy-stearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable.

Polyglycerol Esters

Typical examples of suitable polyglycerol esters are Polyglyceryl-2 Dipolyhydroxystearate (Dehymuls® PGPH), Polyglycerin-3-Diisostearate (Lameform®) TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor®) GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylolpropane or pentaerythritol with lauric acid, cocofatty acid, tallow fatty acid, palmitic acid, stearic acid, oleic acid, behenic acid and the like optionally reacted with 1 to 30 mol ethylene oxide.

Anionic Emulsifiers

Typical anionic emulsifiers are aliphatic fatty acids containing 12 to 22 carbon atoms such as, for example, palmitic acid, stearic acid or behenic acid and dicarboxylic acids containing 12 to 22 carbon atoms such as, for example, azelaic acid or sebacic acid.

Amphoteric and Cationic Emulsifiers

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine. Finally, cationic surfactants are also suitable emulsifiers, those of the esterquat type, preferably methyl-quaternized difatty acid triethanolamine ester salts, being particularly preferred.

Description of the Production Process

To produce the dispersions, a preparation of the oil component and emulsifier is preferably first prepared at ambient temperature. Based on the oil phase, the emulsifier may be present in quantities of 0.5 to 10% by weight, preferably 1 to 5% by weight and more particularly 1.5 to 2% by weight. An aqueous preparation containing the gelatin and the active component(s) is then added dropwise to the oil phase. The content of gelatin and active components is normally of the order—based on the aqueous phase—of 1 to 50% by weight, preferably 10 to 40% by weight and more particularly 15 to 25% by weight. The temperature of the aqueous phase is sufficient to melt the gelatin and is normally in the range from 50 to 90° C. Introduction into the oil phase is generally by dropwise addition, the quantity ratio between the aqueous phase and oil phase being variable from 10:90 to 50:50. In order to promote encapsulation and to prevent particle agglomeration, it is advisable to stir the preparation vigorously, for example at 500 to 1,500 r.p.m., and to cool it below the melting point of the gelatin, i.e. to temperatures of 5 to 10° C., for example by an ice bath. A crosslinking agent is then added to the preparations. Although this step is not essential per se, it has been found that the resulting hardening of the capsules distinctly improves stability in seawater. Suitable crosslinking agents, which are capable of reacting off with the protein matrix of the gelatin capsules, are—above all—short-chain aliphatic aldehydes, such as for example formaldehyde or, more particularly, glutaraldehyde. The crosslinking agents are typically used in quantities of 0.5 to 2% by weight, based on the preparation as a whole. The crosslinking as such may take place at room temperature and takes between 1 and 24 hours, depending on the required hardening level.

After the active components have been encapsulated and the capsules themselves have been hardened, the oil phase is removed and the microcapsules are redispersed in the organic solvent. These two steps are also non-essential, i.e. basically, the aqueous phase may also be directly added dropwise to the organic solvent or the microcapsules need not be redispersed in the organic solvent and may be introduced in the oil phase into the paints. In practice, a small quantity of the later solvent is even added to the oil phase during encapsulation. However, it has been found that, despite intensive shearing, dropwise addition to the organic solvent readily leads to agglomeration and to the formation of irregularly shaped or unsatisfactorily encapsulated aggregates. Conversely, there is a commercial need to use only dispersions of which the solvents are present in any event in commercial paints. The dispersions are added to the paints in quantities of typically 5 to 50% by weight, preferably 10 to 40% by weight and more particularly 15 to 25%% by weight.

Commercial Applications

The dispersions according to the invention are particularly suitable for finishing paints in such a way that they afford the materials thus treated improved protection both against microorganisms and against corrosion factors. Accordingly, the present invention also relates to the use of the dispersions for the production of paints, preferably paints intended for use under water, particularly seawater, the content of the dispersions in the paints being from 1 to 50% by weight, preferably from 5 to 30% by weight and more particularly from 10 to 20% by weight. The present invention also relates to the use of the dispersions for treating or finishing materials such as, for example, wood, steel, concrete or steel-reinforced concrete, for example by impregnation, coating or addition, more especially for protection against fouling and/or corrosion.

### EXAMPLES

3 g gelatin and 2 g copper(II) oxide were introduced into a 100 ml stirred reactor and, after the addition of 17 ml water (phase A), were stirred at a temperature of 65 to 70° C. until a homogeneous suspension was formed. In a second 500 ml stirred reactor, an emulsifier was added to 80 g of an oil component at 20° C. (phase B). The hot aqueous suspension was then stirred into the oil phase which, after about 1 minute, was cooled in an ice bath for 45 minutes to about 10° C., the copper oxide being encapsulated in a gelatin matrix. 1 g glutaraldehyde (phase C) and 30 ml xylene were then added to the oil phase with the microcapsules present therein for the purpose of crosslinking. The preparation was left standing overnight and, on the next day, was freed from the oil phase by decantation. The microcapsules were then re-suspended in xylene. Details of the production of the microcapsules according to Examples 1 to 6 are set out in Table 1 below.

TABLE 1
Production of microencapsulated CuO pigments

| Phase | Component | 1 | 2 | 3 | 4 | 5 | 6 |
|---|---|---|---|---|---|---|---|
| A | Gelatin | 3.0 | | | | | |
| | CuO | 2.0 | | | | | |
| | Texamin ® KE 3160 | 0.4 | | | | | |
| | Coco | | | | | | |
| | monoethanolamide | | | | | | |
| | Water | 17.0 | | | | | |
| B | Spindle ® 90 | 80.0 | — | — | 80.0 | — | 80.0 |
| | Mineral oil | | | | | | |
| | Cetiol ® 868 | — | — | — | — | 80.0 | — |
| | 2-Ethyl- | | | | | | |
| | hexyloctadecanoate | | | | | | |
| | Dub-Mi | — | 80.0 | — | — | — | — |
| | Mineral oil | | | | | | |
| | Cileno | — | — | 80.0 | — | — | — |
| | Mineral oil | | | | | | |
| | Dehymuls ® HRE7 | — | 1.0 | — | — | — | — |
| | PEG7 Hydrogenated | | | | | | |
| | Castor Oil | | | | | | |
| | Dehymuls ® PGPH | — | — | 0.5 | — | — | — |
| | Dipolyglycerol | | | | | | |
| | Polyhydroxystearate | | | | | | |
| | Span ® 80 | 1.5 | — | 0.5 | 1.5 | 1.5 | 1.5 |
| | Sorbitan monooleate | | | | | | |

| | | |
|---|---|---|
| C | Glutaraldehyde | 1.0 |

| | | | | | | |
|---|---|---|---|---|---|---|
| Stirring speed [r.p.m.] | 1000 | 600 | 400 | 600 | 600 | 400 |
| Particle diameter [microns] | 30 | 40 | 40 | 40 | 100 | 100 |

### BRIEF DESCRIPTION OF THE DRAWING

The FIGURE is a Micrograph of the capsules of Example 4.

1: A dispersion comprising:
(a) a microencapsulated anti-fouling component. and
(b) an organic solvent.

2: The dispersion as claimed in claim 1, comprising anti-fouling components encapsulated in a gelatin matrix.

3: The dispersion as claimed in claim 1 comprising microcapsules with a mean diameter of 10 to 150 microns.

4: The dispersion as claimed in claim 1, wherein the anti-fouling component comprises: at least one member selected from the group consisting of inorganic salts, inorganic pigments and organometallic compounds.

5: The dispersion as claimed in claim 1, wherein the anti-fouling component comprises at least one of oxides of copper and oxides of iron.

6: The dispersion as claimed in claim 1, wherein the microcapsules contain corrosion inhibitors as further active components.

7: The dispersion as claimed in claim 1, comprising at least one organic solvent selected from the group consisting of aliphatic, cycloaliphatic and aromatic hydrocarbons.

8: The dispersion as claimed in claim 1, comprising the microencapsulated active components and the organic solvent in a ratio by weight of 10:90 to 90:10.

9: A process for the production of dispersions of microencapsulated active components comprising the steps of:
(a) preparing a first mixture of an oil component and an emulsifier at ambient temperature,
(b) preparing an aqueous second mixture of gelatin and the active components at a temperature in the range from 50 to 100° C.,
(c) adding the aqueous second mixture to the first oil-containing preparation with agitation to form a third mixture,
(d) cooling the third mixture to a temperature below the melting point of the gelatin to form a microcapsule dispersion,
(e) adding a crosslinking agent to the microcapsule dispersion to form crosslinked microcapsules,
(f) removing the oil phase, and,
(g) redispersing the crosslinked microcapsules in an organic solvent.

10: A paint comprising the microcapsule dispersion of claim 1.

11: A method of finishing or treating materials which comprises applying to the materials the microcapsule dispersion of claim 1.

12: The dispersion as claimed in claim 2 comprising microcapsules with a mean diameter of 10 to 150 microns.

13: The dispersion as claimed in claim 2, wherein the anti-fouling component comprises: at least one member selected from the group consisting of inorganic salts, inorganic pigments and organometallic compounds.

14: The dispersion as claimed in claim 2, wherein the anti-fouling component comprises at least one of oxides of copper and oxides of iron.

15: The dispersion as claimed in claim 2, wherein the microcapsules contain corrosion inhibitors as further active components.

16: The dispersion as claimed in claim 2, comprising at least one organic solvent selected from the group consisting of aliphatic, cycloaliphatic and aromatic hydrocarbons.

17: The dispersion as claimed in claim 2, comprising the microencapsulated active components and the organic solvent in a ratio by weight of 10:90 to 90:10.

18: A paint comprising the microcapsule dispersion of claim 2.

19: A method of finishing or treating materials which comprises applying to the materials the microcapsule dispersion of claim 2.

20: The dispersion of claim 1 wherein microcapsules of the microencapsulated anti-fouling component have a mean particle of from 0.1 mm to 1.5 mm.
